(19) United States
(12) Patent Application Publication
(10) Pub. No.: US 2021/0192436 A1
(43) Pub. Date: Jun. 24, 2021

(54) METHODS AND SYSTEMS FOR MANAGING PRODUCT EXPIRATION

(71) Applicant: WAVEMARK, INC., Concord, MA (US)

(72) Inventors: Rahul TIWARI, Arlington, VA (US); Fadi ASSAF, Quebec (CA); Mirella AOUN, Concord, MA (US); Jihad RAHMEH, Beirut (LB); Christina HAMOUSH, Beirut (LB); Rima ABDUL-LATIF, Concord, MA (US)

(21) Appl. No.: 17/125,276

(22) Filed: Dec. 17, 2020

Related U.S. Application Data
(60) Provisional application No. 62/951,758, filed on Dec. 20, 2019.

(51) Int. Cl. G06Q 10/08; G06K 19/077; G06N 20/00

(52) U.S. Cl. CPC G06Q 10/087; G06N 20/00; G06K 19/07758

(57) ABSTRACT

A radio frequency identification (RFID) tag affixed to a product, and systems and methods for managing tag and associated product information and use thereof. The system may include a RFID reader configured to acquire data from the product affixed with the RFID tag, in which the data corresponds to characteristic information regarding the product, as well as other information related to the product, such as location related information. The system may also comprise a server configured to receive the data from the RFID reader, in which the server stores the received data with additional information regarding the product in a memory. The system may also comprise an analyzation unit configured analyze the stored data and previously stored data, including via use of machine learning and/or analysis algorithms and features. The system may also comprise a notification unit configured to generate an alert based on the analyzation of the stored data and the previously stored data.

### CROSS-REFERENCE TO RELATED APPLICATIONS

This application claims priority to U.S. Provisional Application No. 62/951,758, filed Dec. 20, 2019. The disclosure of the priority application is hereby incorporated in its entirety by reference.

### TECHNICAL FIELD

Aspects of the present disclosure are directed to systems and methods for managing the expiration of products and the supply chain thereof.

### BACKGROUND AND SUMMARY

This background and summary are provided to introduce a selection of concepts in a simplified form that are further described below in the DETAILED DESCRIPTION. This background and summary are not intended to identify key features of the claimed subject matter, nor are they intended to be used as an aid in determining the scope of the claimed subject matter.

Medical products, for example, surgical devices, drugs, medical devices, medical supplies, etc. (hereinafter also individually and collectively interchangeably referred to herein as “medical products”), may be provided to customers (hereinafter also interchangeably referred to herein as “consumers”), for example, hospitals, doctors' offices, pharmacies, etc., to be consumed by and/or used with respect to medical procedures on patients. Based on many outside factors, examples of which are discussed below, these medical products may not be used at the customer location during their efficacy period; for example, the medical products may remain in storage, on the shelves, or elsewhere at the customer location without being prescribed to patients. Once these medical products reach their expiration dates (hereinafter also interchangeably referred to herein as “expired products”), they may be discarded, destroyed, sent back to the manufacturer to be refurbished, or, in an example worst case scenario, used by patients. The use of expired products may put patients at risk, and may indirectly cost customers significant amounts of money if, for example, a patient has an adverse reaction to the use of an expired product. The discarded or destroyed expired products may cause loss of revenue to consumers because the expired product should not have been used. Further, the expired products that are subject to refurbishment may also cause loss of revenue to consumers, as the process of refurbishment may be expensive. Clinical and supply chain executives are challenged with managing supplies to support patient safety, drive efficient workflows, reduce loss of revenue, ensure customer locations are properly equipped, and minimize waste. Each year roughly 8.5% of medical products turn into expired products, which results in millions of dollars in lost revenue, among other losses and problems.

To automate a supply chain of products, radio frequency identification (RFID) tags are frequently used to identify and track medical products. For example, RFID tags may be attached to some medical products for purposes of tracking. RFID tags may uniquely identify their host product using a pre-programmed tag identifier (TID), which may be a unique serial number assigned by the chip manufacturer. The RFID tags may include a memory bank to store items' unique tracking identifiers, such as electronic product codes (EPCs). In some aspects, additional information may be stored directly in the memory bank or a secondary memory bank of an RFID tag. The additional information may include, for example, a product code, lot number, and expiration date of a product associated with the RFID tag.

Common types of RFID tags include low frequency (LF), high frequency (HF) and ultra-high frequency (UHF) RFID tags. LF RFID tags generally operate at a frequency of about 30 KHz to 300 KHz, and may only be scanned by a reader within extremely close proximity to the LF RFID tag, e.g., approximately less than 10 cm. HF RFID tags generally operate at a frequency of about 3 to 30 MHz, and may only be scanned by a reader within relative close, but not necessarily extremely close, proximity to the HF RFID tag, e.g., approximately between 10 cm and 1 m. UHF RFID tags generally operate at a frequency of about 300 MHz to 3 GHz, and may be scanned from a greater distance than LF and HF RFID tags, e.g., a distance of up to approximately 12 m. Inventory tracking systems may be based on LF RFID tags, HF RFID tags, UHF RFID tags, barcodes, etc., for example.

Aspects of the present disclosure relate to systems and methods for managing the expiration dates of inventory affixed with RFID tags and/or barcodes, among other types of product identification elements. Such systems and methods may include one or more devices, such as one or more computers or other terminal devices and/or computer systems for managing inventory through the supply chain, and/or managing the expiration dates of the inventory in order to ensure the medical products are, inter alia, consumed and/or adjusted via relocation prior to the expiration date. The system may include features for applying machine learning algorithms to provide end to end real time enterprise visibility that, among other advantages, may help clinical and supply chain executives to reduce product expiration and waste, thereby reducing overall lost profits of the company.

Additional advantages and novel features of these aspects will be set forth in part in the description that follows, and in part will become more apparent to those skilled in the art upon examination of the following or upon learning by practice of the disclosure.

### BRIEF DESCRIPTION OF THE DRAWINGS

The novel features believed to be characteristic of implementations of the disclosure are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further features and advances thereof, will be best understood by reference to the following detailed description of illustrative implementations of the disclosure when read in conjunction with the accompanying drawings, wherein:

FIG. 1 illustrates an example network for managing inventory through the supply chain and lifecycle of a product in accordance with aspects of the present disclosure;

FIG. 2 illustrates an example storage cabinet in accordance with aspects of the present disclosure;

FIG. 3 illustrates a flowchart of one example implementation for managing inventory through the supply chain and expiration of a product in accordance with aspects of the present disclosure;

FIG. 4 illustrates various features of an example computer system for use in conjunction with aspects of the present disclosure; and

FIG. 5 illustrates a block diagram of various example system components, in accordance with aspects of the present disclosure.

### DETAILED DESCRIPTION

The following includes definitions of selected terms employed herein. The definitions include various examples and/or forms of components that fall within the scope of a term and that may be used for implementation. The examples are not intended to be limiting.

A “processor,” as used herein, processes signals and performs general computing and arithmetic functions. Signals processed by the processor may include digital signals, data signals, computer instructions, processor instructions, messages, a bit, a bit stream, or other computing that may be received, transmitted and/or detected.

A “bus,” as used herein, refers to an interconnected architecture that is operably connected to transfer data between computer components within one or more systems. The bus may be a memory bus, a memory controller, a peripheral bus, an external bus, a crossbar switch, and/or a local bus, among others. The bus may also be a bus that interconnects components inside a system using protocols, such as Controller Area network (CAN), Local Interconnect Network (LIN), among others.

A “memory,” as used herein may include volatile memory and/or non-volatile memory. Non-volatile memory may include, for example, ROM (read only memory), PROM (programmable read only memory), EPROM (erasable PROM) and EEPROM (electrically erasable PROM). Volatile memory may include, for example, RAM (random access memory), synchronous RAM (SRAM), dynamic RAM (DRAM), synchronous DRAM (SDRAM), double data rate SDRAM (DDR SDRAM), and/or direct RAM bus RAM (DRRAM).

An “operable connection,” as used herein may include a connection by which entities are “operably connected,” is one in which signals, physical communications, and/or logical communications may be sent and/or received. An operable connection may include a physical interface, a data interface and/or an electrical interface.

A “wired or wireless connectivity,” as used herein may include, but not be limited to one or more universal serial bus (USB) connections, wireless fidelity (“Wi-Fi”) coupling, Bluetooth or Bluetooth Low Energy (BLE) coupling, Ethernet connection, cable connection, digital subscriber line (DSL) connection, cellular coupling (e.g., 3G, LTE/4G or 5G), or other suitable coupling or couplings.

Generally described, aspects of the present disclosure provide systems and methods for managing the expiration of product and the supply chain thereof. For instance, an example system in accordance with aspects of the present disclosure may provide for seamless visibility of products flowing from supply chain to usage/consumption. This visibility may be achieved using a RFID tag that may provide analytics and insights into the supply chain and lifecycle of products having attached thereto or being associated with RFID tags, among other features. Additionally, the example system may track a variety of products from various origins and points of entry into the system having a variety of RFID tag types affixed to or associated with the products. That is, the system may track products having any one of a combination of LF, HF, and/or UHF RFID tags. Furthermore, the example system may facilitate seamless inventory tracking and reporting. Types of products that may be managed through the supply chain and lifecycle via the use of RFID tags, among other types of product identification elements, may include, for example, medical equipment, medical devices, pharmaceuticals, consumable goods, and the like.

RFID tags, also interchangeably referred to herein as “tags,” generally take the form of integrated circuits, with associated antennae, that have computer readable memories that may be encoded with identification information, such as a unique serial number (USN), which may also be referred to interchangeably herein as a unique identification number, unique digital identifier, universal identifier, or “UID.” The identification information may include information about the tag itself, such as the manufacturer of the tag, date of manufacture, lot number, tag configuration, expiration date, if the product the tag is affixed to may be refurbished, how many times the product the tag is affixed to is refurbished, etc. Typically, when an RFID tag is interrogated, the tag responds by emitting a data signal that includes the tag's UID, which is captured by the reader. This technique and other examples of similar techniques for managing inventory through the supply chain and lifecycle of a product are described, for example, in further detail in U.S. patent application Ser. No. 16/543,246, filed on Aug. 16, 2019, to Leitermann et al., which is herein incorporated by reference in its entirety.

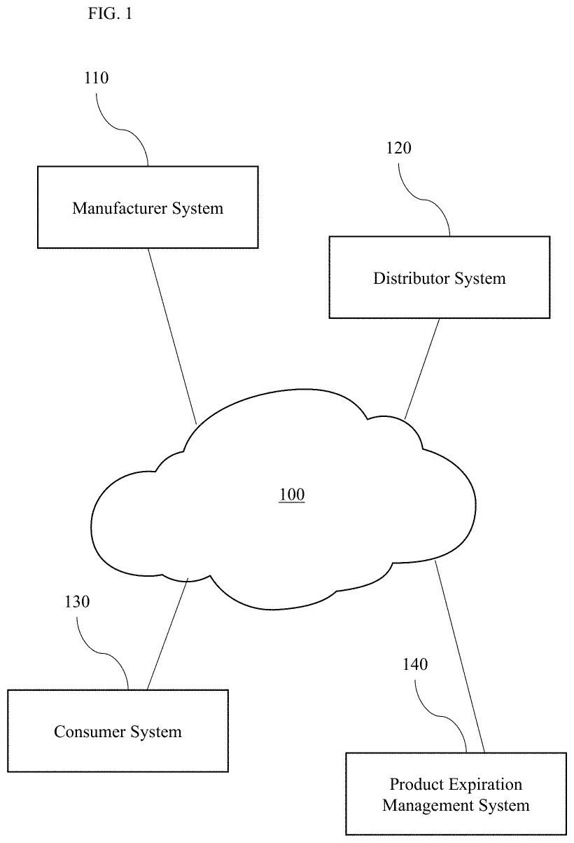

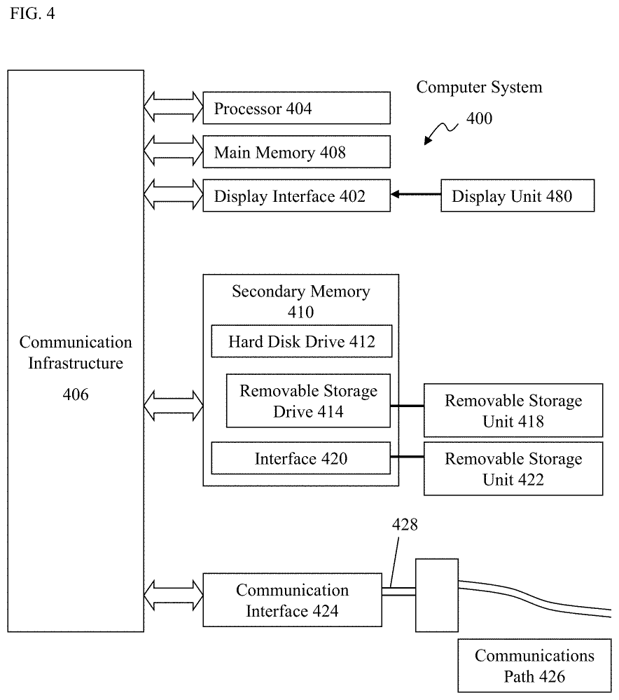

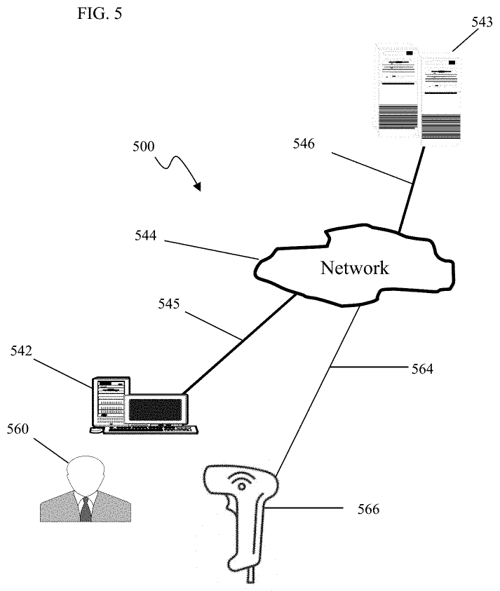

Referring now to FIG. 1, therein illustrated is an example network 100 that may incorporate and/or communicate with and among various other features for managing inventory, the expiration of products comprising the inventory, and the supply chain thereof, as well as other activities, in accordance with aspects of the present disclosure. For example, the network 100 may include or be coupled with a manufacturer system 110, a distributor system 120, a consumer system 130, and a product expiration management system 140. For example, network 100 may thereby be used to facilitate communications among multiple systems, including the manufacturer system 110, the distributor system 120, the consumer system 130, and the product expiration management system 140. In some implementations, some or all of the components illustrated in FIG. 1 may be in a single general physical location or may be in or include one or more remote locations (e.g., may be cloud based). For example, the manufacturer system 110, the distributor system 120, and the consumer system 130 may be located in the same or different physical locations, and the product expiration management system 140 may be located in the cloud, and accessed by users at different locations, as shown with respect to FIG. 5, for example, and described in more detail conjunction therewith. In some implementations, the network 100 may include the Internet or another Internet Protocol (IP) based network. The manufacturer system 110, the distributor system 120, the consumer system 130, and the product expiration management system 140 may include one or more computer systems, which may include one or more terminals having various features as shown with respect to FIG. 4 and FIG. 5, for example, and described in more detail in conjunction therewith. In some implementations, the manufacturer system 110, the distributor system 120, the consumer system 130, and the product expiration management system 140 may also include a memory that stores instructions for executing processes for managing inventory through the supply chain and lifecycle of a product, and a processor configured to execute the instructions.

In some implementations, the product expiration management system 140 may track one or more products tagged with an RFID tag through the supply chain, for example, from manufacture to expiration of the product. For example, the supply chain may include various activities, phases and/or features in the product lifecycle, including for example, manufacturing, packaging, transportation, distribution, inventory, usage/consumption, refurbishment/reprocessing, expiration and/or disposal, as described in more detail below. Some or all of these activities, phases, and/or features of the supply chain may be associated with a location or feature equipped with one or more different types of RFID reader devices. Some examples of RFID readers include handheld scanners for users to scan locations, fixed scanners located within a device to scan specific locations, and fixed scanners within large locations to scan large locations, as described in more detail below. In order to afford compatibility with a number of different types of RFID reader devices, a number of different types of RFID tags, and/or a number of different operating environments, the products may be tracked using multiple different RFID protocols, thereby enabling seamless item-level identification and management/tracking of the products throughout the supply chain, for example. The different RFID protocols may generally use one or more frequency bands, which may be referred to generally as LF, HF, and UHF.

In some implementations, the RFID tag may be provided to the product's manufacturer by a purveyor of the product expiration management system 140. For example, the RFID tag may be applied by the manufacturer to a product using an applicator device that may uniquely register or position the RFID tag onto a specific location on a package during affixation. In another aspect of the disclosure, the RFID tag may be provided by the purveyor of the product expiration management system 140, and the tag may be affixed to the product at any point in the product's life cycle, for example, at the distributor, consumer site, etc. This process of affixing and registering the RFID tag may be conducted via one or more of the systems shown in FIGS. 4 and 5, as described in more detail below. In some implementations, the RFID tag may be applied to a packaging of a product rather than the physical product itself at any time during the product's life cycle.

As described above, in some implementations, the RFID tag that is provided by the purveyor of the product expiration management system 140 may be applied to the product itself or the packaging of the product during any point in the product's lifecycle. In one aspect of the disclosure, the RFID tag may be applied at any point during the manufacture process, the distribution process, and/or the consumption process. Upon affixing the RFID tag to the product or packaging of the product, the RFID tag may be read by an RFID reader, as described below, which obtains a UID, and the UID may be registered in a respective database, along with other characteristic information, for example, the expiration date of the product. In one aspect, if a RFID tag is registered during the manufacturing process, the RFID tag's UID may be registered at the manufacturer system 110. As described above, each system 110, 120, 130 may include its own database, and each system may be implemented with different RFID readers operating on different frequencies, for example. For example, the manufacturer may manufacture a stent bearing serial number 0001 and having an expiration date of May 1, 2020. The manufacturer may register the UID of the RFID tag in the manufacturer system 110 along with information indicating the serial number and the expiration date of the stent.

Further, the distributor may obtain the stent from the manufacturer and may be able to use a RFID reader to read the same UID from the RFID tag affixed to the stent's packaging. Further, the distributor system 120 may correlate the UID read from the RFID tag with additional characteristic information provided by the RFID reader, and store this additional characteristic information in the database along with the UID. For example, the distributor system may store “UID 0005” along with information indicating that the product associated with the tag bearing UID 0005 was received at 06:07 PM on Jan. 1, 2019, and shipped to a consumer at 07:05 PM on Mar. 2, 2020.

As described in the example above, although the distributor system 120 may not have access to the manufacturer system 110 containing the database with the UID and the additional characteristic information, the distributor system may register the example UID 0005 in the distributor system 120 containing its own database. As described below, each discrete database stored on a respective system may or may not be shared among systems 110, 120, 130 and 140, and/or may only be shared from 110, 120, and/or 130 with system 140.

In one aspect of the disclosure, for example, the manufacturer system 110 that registered and stored UID 0005 as a stent with a specific expiration date of May 1, 2020, may communicate with distributor system 120. Upon receipt of the product by the distributor from the manufacturer, the RFID tag comprising the same UID 0005 is read through the use of a RFID reader. The distributor system 120 may query manufacturer system 110 and determine that UID 0005 is registered as a stent with an expiration date of May 1, 2020. The distributor system may, for example, register UID 0005 in the distributor system 120 database as a stent that was received and had its tag read at 06:07 PM on Jan. 1, 2019. In another example, distributor system may, for example, update UID 0005 in the manufacturer system 110 database as being received by the distributor and having had its tag read at the distributor at 06:07 PM on Jan. 1, 2019.

In another aspect of the disclosure, each system 110, 120 and 130 may communicate with each other and/or with product expiration management system 140. For example, the manufacturer system 110 may register and store UID 0005 as a stent with an expiration date of May 1, 2020, in its own database, but also communicate that information to the product expiration management system 140. Upon receipt of the physical product by the distributor from the manufacturer, the RFID tag comprising the same UID 0005 may be read, as described above, and the distributor system 120 may query the product expiration management system 140 and determine that UID 0005 is registered as a stent with an expiration date of May 1, 2020. The distributor system may, for example, register UID 0005 in the distributor system 120 database as a stent that was received and read at 06:07 PM on Jan. 1, 2019. In another example, the distributor system 120 may, for example, update UID 0005 in the product expiration management system 140 database as being received at the distributor at 06:07 PM on Jan. 1, 2019. Thus, each system 110, 120 and 130 may, for example, locally store its own data in a database without interaction with any other databases contained within the systems of FIG. 1, locally store its own data in a database with data sharing enabled among systems, locally store in its own database with data sharing among systems with data editing capability, or locally store its own data in a database and share data with the product expiration management system 140 only, etc. In some aspects of the disclosure, all or some of the data may be written directly onto a memory bank of the RFID tag such that the data can be read directly from the RFID tag. For example, upon associating a RFID tag with a product, the manufacturer may write the product's product code, serial number, and expiration date to the memory bank of the RFID tag. Upon receiving the product, the manufacturer can read the tag's UID and can also read the product code, serial number, and expiration date directly from the tag's memory bank without accessing the manufacturer system. In some aspects, some or all of the data stored in the RFID tag's memory bank is encrypted and/or read/write access to the data or memory bank is restricted to prevent unauthorized access, use, or modification of the data or memory bank. In one aspect of the disclosure, the product expiration management system 140, described above, may comprise data from a wide variety of customers. For example, the product expiration management system 140, may comprise data of over 250 customers, for example, hospitals, and at any given time, comprising, for example, up to 10 million or more different UIDs. The data corresponding to each of the UIDs, as described above, may provide, for example, the location of a product associated with the RFID tag comprising the specific UID, the expiration date of the product, the date and time the product is used/consumed, the quantity of similar products, the quantity of similar products with each customer, etc. The use of the data stored by the product expiration management system 140, is described in more detail below.

In another aspect of the disclosure, the product expiration management system 140 may implement machine learning algorithms and/or apply other methods of analysis on the data/information shared among one or more of the systems 110, 120 and 130, as described above, and stored on or accessed by the product expiration management system 140, described in more detail below.

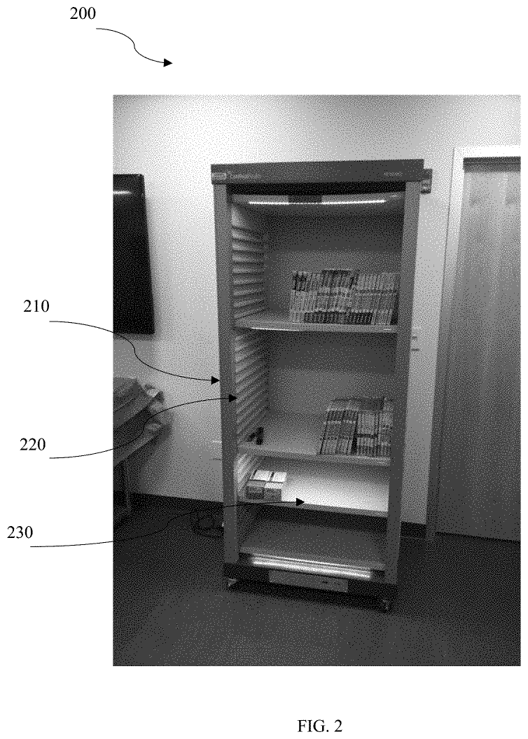

Referring now to FIG. 2, therein illustrated is an example device in accordance with aspects of the present disclosure. In one aspect, the storage cabinet 200 may include various RFID enabled features contained within the storage cabinet 200. For example, as described in more detail below, the storage cabinet 200 may include or interoperate with a plurality of RFID enabled readers. In one aspect of the disclosure, the plurality of RFID enabled readers may comprise a plurality of antennae embedded or attached to the shelves, walls, top, bottom, etc., of the storage cabinet 200, for example, including a plurality of antenna loops. As discussed in more detail below, these antennae may be overlapping with one another, thereby creating a plurality of antenna loops configured to read RFID tags.

In one aspect of the disclosure, the storage cabinet 200 may be configured to be implemented by the consumer system 130, referenced in FIG. 1, and described in more detail above. In another aspect of the disclosure, handheld RFID readers, or whole room RFID readers may be configured to be to implemented with manufacturer system 110 and/or distributor system 120 referenced in FIG. 1, described in more detail above. Although the storage cabinet 200 and handheld readers may be referenced as RFID reader(s), any suitable type of the RFID reader(s) may obtain data and/or register a product affixed with a RFID tag. Further, the data obtained by a RFID reader may be provided to the product expiration management system 140, referenced in FIG. 1, and described above.

In another aspect of the disclosure, the storage cabinet 200 may be mobile, such that it may be relocated, rather than being permanently affixed to a single location after installation. Further, the storage cabinet 200 may be placed, for example, within close proximity to a second storage cabinet 200. For example, the storage cabinet 200 may abut, be located next to, back-to-back with, or in close proximity to a second storage cabinet, for example, in the same room. In one aspect of the disclosure, the storage cabinet 200 may include a housing 210 having a plurality of slots or other shelf retaining features 220 that are configured to support and/or provide communications with a respective shelf 230 supported by or in proximity thereof. In this manner, the number of shelves and spacing between each pair of successive shelves implemented in the storage cabinet 200 may be customizable, for example, based on user needs and/or a product size stored thereon. For example, the shelf 230 may have additional shelves placed immediately above and/or below the area containing slots 220. This technique and other examples of similar techniques for storing and/or reading RFID tags are described in further detail in U.S. Provisional Patent Application No. 64/726,635, filed on Nov. 21, 2018, to Leitermann et al., which is herein incorporated by reference in its entirety.

In another aspect of the disclosure, the products affixed with the RFID tag may be stored, for example, in a storage room, an RFID-enabled storage shelf, cabinet, or cold-storage space, etc., until needed. Additionally, while the products are stored by a consumer, the RFID tag may be scanned by the consumer system 130, as referenced in FIG. 1, and such information may be transmitted to the product expiration management system 140, as also referenced FIG. 1. In this way, the product expiration management system 140, as referenced in FIG. 1, may monitor each of the products stored by the consumer to determine whether there are expired products, and/or products near expiration, for example. In some implementations, upon determining that a given product is about to expire or is expired, the product expiration management system 140, as referenced in FIG. 1, may monitor the consumer system 130, as also referenced in FIG. 1, to determine the next steps to be taken for the product that is about to expire or is expired, for example, by sending a notification to the consumer via an alert, moving the product to a different location, reviewing historical data of the product, reviewing other locations of the consumer based system 130, etc., as described in more detail below.

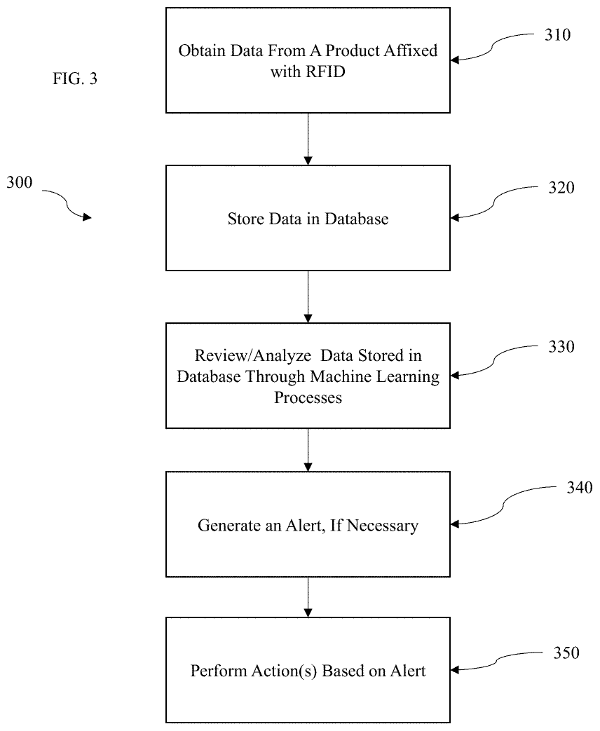

FIG. 3 illustrates one example implementation of a method for managing the expiration of a product through a supply chain. The method may include reviewing and analyzing current and historical data patterns and/or trends of the lifecycle of products affixed with RFID tags. For example, the product expiration management system 140, of FIG. 1 described in more detail above, may determine that product X, affixed with RFID tag “UID 000A” is expiring next week, and thus the product should be used/consumed as soon as possible to prevent loss of revenue to the consumer. Method 300, as described below, may be used to determine why the product was not used/consumed prior to the expiration date, how to use/consume the product prior to the expiration date, and how to correct the problem of ensuring that a similar product in the future is not left to expire.

Referring to method 300 of FIG. 3, at block 310 the system receives data regarding a product affixed with a RFID tag. For example, as discussed above in relation to FIGS. 1 and 2, a product may be registered in the product expiration management system 140 (FIG. 1), along with corresponding data regarding the product, for example, the location of the consumer where the product was received, the specific location of the product within the location of the consumer, the time the product was received, the type of product, and the product expiration, among other data. For example, tag UID 000A may be associated with a stent that may be received by hospital A, stored in cabinet B on shelf 1, at time 12:00 PM ET on Jan. 1, 2019, with an expiration date of Jan. 1, 2020.

At step 320, the data received by reading the RFID tag affixed to the product may be stored in a database. For example, as described in more detail above, the data may be obtained by any of the systems with relation to FIG. 1, and the data may be stored by any of the systems, for example 110, 120, 130, and 140. For example, with reference to FIG. 1, the consumer system 130, located at hospital A, may receive the product affixed with the RFID and may scan the RFID tag to obtain the data, as discussed in more detail above. The data obtained by the consumer system 130 may be processed and transmitted via the network 100 to the product expiration management system 140 for storing.

At block 330, the system may continuously review and/or analyze the data as it is being stored, and may also review and/or analyze previously stored data in the database. In one example in accordance with aspects of the present disclosure, the process of review and or analysis may be performed using machine learning algorithms. A variety of machine learning algorithms may be implemented with regard to the stored data, but the system may only report the results of a single algorithm (e.g., the most accurate algorithm), for example. In an aspect, the product expiration management system 140 may continuously review the data provided to the system for storing, along with data previously stored in the system, for example, to determine if a product is likely to expire prior to being used/consumed, using the machine learning algorithms. For example, the product expiration management system 140, referenced in FIG. 1, may review the data stored in the database and determine that hospital A has 500 units of product X in stock, and that 200 of these units may be nearing their expiration date, such as within a month, for example. Using previously stored data and machine learning algorithms, the product expiration management system 140 may also take into account the typical usage rate of product X at hospital A, which may indicate that only 50 units of product X are typically used/consumed per month. Based on this analysis, the hospital may have 150 units likely to expire without being used/consumed, and thus the system may generate an alert, as described in more detail below, indicating that an action should be taken regarding the 150 units of product X likely to expire without being timely used. In one aspect of the disclosure, the machine learning algorithms may be continuously updated based on data provided on a lifecycle of a product and/or location, and/or consumer. In another aspect of the disclosure, the machine learning algorithms may be implemented to forecast if and when products may expire prior to being used/consumed.

In another example with regard to block 330 of FIG. 3, in accordance with aspects of the disclosure, the system may continuously review and/or analyze the data currently being stored, along with previously stored data in the database, for example, to determine if a product has a short remaining product life (also interchangeably referred to herein as a “short-dated product”). Such short-dated products may be products provided to a consumer that have a less than an industry standard remaining life to expiration date. For example, data provided to the product expiration management system 140 in FIG. 1 may contain data that product X, a stent, was received by hospital A, stored in cabinet B on shelf 1, at time 12:00 PM ET on Jan. 1, 2019, with an expiration date of Jan. 5, 2019. Thus, product X was provided to hospital A only 4 days prior to its expiration date. In one aspect of the disclosure, the product expiration management system 140, may determine that this product received by the manufacturer is a short-dated product. The system may generate an alert, as described below, indicating the product that was recently received by the consumer location is short-dated.

In another example with regard to block 330 of FIG. 3, in accordance with aspects of the present disclosure, the system may continuously review and/or analyze the data currently being stored, along with previously stored data in the database, for example, to determine if a product is a non-moving product. For example, data provided to the product expiration management system 140 may indicate that hospital A has 10 units of product X. Based on historical data, hospital A has ceased using product X, and none of these products are likely to be used/consumed by the consumer location in the relative future. The system may generate an alert, as described in more detail below, indicating the product may expire because it may not be used/consumed by the consumer at the product's present location.

In another example with regard to block 330 of FIG. 3, in accordance with aspects of the present disclosure, the system may continuously review and/or analyze the data currently being stored, along with previously stored data in the database, for example, to determine if a consumer location is consistent with or fails to utilize products in a first in, first out order (FIFO). For example, data provided to the product expiration management system 140 in FIG. 1 may indicate that product X, a stent, was received by hospital A, stored in cabinet B on shelf 1, at time 12:00 PM ET on Jan. 1, 2019, with an expiration date of Jan. 5, 2019. The product expiration management system 140, may also determine that Y, a stent, which was received by hospital A, stored in cabinet B on shelf 2, at time 12:00 PM ET on Jan. 2, 2019, with an expiration date of Feb. 5, 2020, was recently consumed before the older stocked product having an earlier expiration date. The system may generate an alert, as described in more detail below, indicating the product X may expire because it will not be consumed based upon the lack of a FIFO usage/consumption order.

In another example with regard to block 330 of FIG. 3, in accordance with aspects of the present disclosure, the system may continuously review and/or analyze the data currently being stored, along with previously stored data in the database, for example, to determine if a product is overstocked. For example, data provided to the product expiration management system 140 in FIG. 1 may indicate that hospital A has 10 units of product X with an expiration date of 6 months. Hospital A may place another order for 10 units of product X. Based on historical data, it may be determined that hospital A typically uses roughly 9 units of product X every 6 months, and thus, there may be need to determine if a duplicate order or unnecessary has occurred. The system may generate an alert, as described in more detail below, indicating the products may expire because an overstock order is being placed by the consumer.

At block 340 of FIG. 3, an alert may be generated by the system indicating that an action may be necessary by the consumer. In one aspect of the disclosure, for example, the alert may be or include an onscreen popup in real-time on a user-interface (UI) of an RFID reader, such as a mobile RFID reader, as described in reference to FIGS. 4 and 5 below, or a status message associated with a line item of a product expiration management system 140 (FIG. 1), which may be displayed on a UI of a computing device, such as a mobile device, laptop, or desktop computer, server, etc., as described in more detail in reference to FIGS. 4 and 5 below. In another aspect of the disclosure, the alert may be provided to a user in an inventory report that may be generated periodically, e.g., daily, weekly, monthly, or as requested by the user, or in real-time as a notification.

At block 350 of FIG. 3, based on the alert, for example, a user may be required to perform an action. In one aspect of the disclosure, for example, the alert may indicate suggested a specific task a user should perform. In another aspect of the disclosure, the alert may require a mandatory action by the user. In yet another aspect of the disclosure, the alert may initiate an action with or without the interaction by the user. For example, as described in more detail above, if the product expiration management system 140 of FIG. 1 determines that the product received by the consumer is short-dated, the alert may indicate that product X should be sent back to the manufacturer. For example, an alert may be generated to a user via the UI, described above (block 340 of FIG. 3), and the user may take any necessary steps to send the product back to the manufacturer (block 350 of FIG. 3). In another example, the user may completely disregard the suggested alert by taking no action and allowing the product to expire at the consumer location. In another example, the alert may trigger a device to initiate shipping of the product back to the manufacturer sua sponte. As described in more detail above, machine learning algorithms may determine the correct action to be taken based upon the alert. For example, the product expiration management system 140 of FIG. 1 may determine that although the product is short-dated, it is likely that the product may be used prior to the expiration date based on a high volume of the specific product being used/consumed within the last week, for example. Thus, in view of the machine learning algorithm, a decision may be made that the product should not be sent back.

In another aspect of the disclosure, through the use of machine learning algorithms with access to data from a network of consumer locations, the system may make a determination to relocate the product to another location within the same consumer location or to another consumer location. For example, the product expiration management system 140 of FIG. 1 may determine that hospital A has quantities of product X stored in operating room 1 that may be expiring in a month. The alert (block 340 of FIG. 3) may indicate that operating room 2 uses large quantities of product X and that a user should relocate quantities of the product from operating room 1 to operating room 2. In another aspect of the disclosure, for example, the product expiration management system 140 of FIG. 1 may determine that hospital A has 5 units of product X that may be expiring in a month. The alert may indicate that hospital B uses large quantities of product X and that a user should relocate all 5 units of product X from hospital A to hospital B in an attempt to use/consume product X before expiration. In another example in accordance with aspects of the present disclosure, the product expiration management system 140 of FIG. 1 may determine that hospital A has ordered 50 units of product X from a manufacturer, but prior to issuing the order to the manufacturer, the system 140 may determine that hospital B has 200 units of product X, but may only consume 10 units per month, for example. The alert (block 340 of FIG. 3) may indicate that hospital B should ship 50 units of product X to hospital A, and may cancel the order to the manufacturer. Based on experimentation, if users perform the action indicated by the alert, expiation of products may be reduced from the national average of about 8.5% to 1-2%, for example.

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. An example of such a computer system 400 is shown in FIG. 1.

Computer system 400 includes one or more processors, such as processor 404. The processor 404 is connected to a communication infrastructure 406 (e.g., a communications bus, cross-over bar, or network). Various software implementations are described in terms of this example computer system. After reading this description, it will become apparent to a person skilled in the relevant art(s) how to implement implementations of the disclosure using other computer systems and/or architectures.

Computer system 400 may include a display interface 402 that forwards graphics, text, and other data from the communication infrastructure 406 (or from a frame buffer not shown) for display on a display unit 480. Computer system 400 also includes a main memory 408, preferably random access memory (RAM), and may also include a secondary memory 410. The secondary memory 410 may include, for example, a hard disk drive 412, and/or a removable storage drive 414, representing a floppy disk drive, a magnetic tape drive, an optical disk drive, a universal serial bus (USB) flash drive, etc. The removable storage drive 414 reads from and/or writes to a removable storage unit 418 in a well-known manner. Removable storage unit 418 represents a floppy disk, magnetic tape, optical disk, USB flash drive etc., which is read by and written to removable storage drive 414. As will be appreciated, the removable storage unit 418 includes a computer usable storage medium having stored therein computer software and/or data.

Alternative implementations of the present disclosure may include secondary memory 410 and may include other similar devices for allowing computer programs or other instructions to be loaded into computer system 400. Such devices may include, for example, a removable storage unit 422 and an interface 420. Examples of such may include a program cartridge and cartridge interface (such as that found in video game devices), a removable memory chip (such as an erasable programmable read only memory (EPROM), or programmable read only memory (PROM)) and associated socket, and other removable storage units 422 and interfaces 420, which allow software and data to be transferred from the removable storage unit 422 to computer system 400.

Computer system 400 may also include a communications interface 424. Communications interface 424 allows software and data to be transferred between computer system 400 and external devices. Examples of communications interface 424 may include a modem, a network interface (such as an Ethernet card), a communications port, a Personal Computer Memory Card International Association (PCMCIA) slot and card, etc. Software and data transferred via communications interface 424 are in the form of signals 428, which may be electronic, electromagnetic, optical or other signals capable of being received by communications interface 424. These signals 428 are provided to communications interface 424 via a communications path (e.g., channel) 426. This path 426 carries signals 428 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage unit 418, a hard disk installed in hard disk drive 412, and signals 428. These computer program products provide software to the computer system 400. Implementations of the present disclosure are directed to such computer program products.

Computer programs (also referred to as computer control logic) are stored in main memory 408 and/or secondary memory 410. Computer programs may also be received via communications interface 424. Such computer programs, when executed, enable the computer system 400 to perform the features in accordance with implementations of the present disclosure, as discussed herein. In particular, the computer programs, when executed, enable the processor 404 to perform the features in accordance with implementations of the present disclosure. Accordingly, such computer programs represent controllers of the computer system 400.

In an aspect of the present disclosure where the disclosure is implemented using software, the software may be stored in a computer program product and loaded into computer system 400 using removable storage drive 414, hard drive 412, or communications interface 420. The control logic (software), when executed by the processor 404, causes the processor 404 to perform the functions described herein. In another aspect of the present disclosure, the system is implemented primarily in hardware using, for example, hardware components, such as application specific integrated circuits (ASICs). Implementation of the hardware state machine so as to perform the functions described herein will be apparent to persons skilled in the relevant art(s).

FIG. 5 is a block diagram of various example system components, for use in accordance with aspects of the present disclosure. FIG. 5 shows a communication system 500 including one or more accessors 560 (also referred to interchangeably herein as one or more “users”), one or more terminals 542 and one or more peripheral input devices 566. Terminal 542 and peripheral input device 566 may include or be located within systems 110, 120, 130 and 140 (FIG. 1), as described above, or within a related or other system in communication therewith, and/or the like. In one aspect, data for use in accordance with aspects described herein may be input and/or accessed by accessors 560 via terminal 542, or peripheral input device 566, such as personal computers (PCs), minicomputers, mainframe computers, microcomputers, telephonic devices, or wired/wireless devices, such as personal digital assistants (“PDAs”) and RFID readers (e.g., handheld, mobile, cabinets, etc.) coupled to a server 543 (e.g., such server 543 may reside within one or more of systems 110, 120, 130 and 140 of FIG. 1), such as a PC, minicomputer, mainframe computer, microcomputer, or other device having a processor and a repository for data and/or connection to a repository for data, via, a network 544 for instance, such as the Internet or an intranet, and couplings 545, 546, 564. The terminal 542 and/or peripheral input device 566 may be used to “register,” add or scan the RFID tag to the systems, described above. Further, the terminal 542 peripheral input device 566 may be implemented to monitor, remove, add, scan, etc. the RFID tags of the system described above. The couplings 545, 546, 564 may include wired, wireless, or fiberoptic links. In another example variation, the method and system in accordance with aspects described herein operate in a stand-alone environment, such as on a single terminal.

The aspects discussed herein may also be described and implemented in the context of computer-readable storage medium storing computer-executable instructions. Computer-readable storage media includes computer storage media and communication media, and may be, flash memory drives, digital versatile discs (DVDs), compact discs (CDs), floppy disks, and tape cassettes. Computer-readable storage media can include volatile and nonvolatile, removable and non-removable media implemented in any method or technology for storage of information such as computer readable instructions, data structures, modules or other data.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of the processes/flowcharts disclosed is an illustration of example approaches. Based upon design preferences, it is understood that the specific order or hierarchy in the processes/flowcharts may be rearranged. Further, some features/steps may be combined or omitted. The accompanying method claims present elements of the various features/steps in a sample order, and are not meant to be limited to the specific order or hierarchy presented.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

What is claimed is:

1. A system for managing expiration dates, comprising:
a radio frequency identification (RFID) reader configured to acquire identification data from a RFID tag associated with a product, wherein the identification data corresponds to information regarding the product;
a server configured to receive the data from the RFID reader, wherein the server stores the received identification data with additional information regarding the product in a data repository;
an analyzation engine configured to analyze the stored data and previously collected or stored data; and
a notification engine configured to generate a status indicator based on the analyzation of the stored data and the previously collected or stored data.

2. The system of claim 1, wherein the additional information includes an expiration date of the product associated with the RFID tag.

3. The system of claim 2, wherein the previously collected or stored data includes usage data with respect to a plurality of products.

4. The system of claim 3, wherein the analyzation engine is configured to apply a machine learning algorithm to the stored data and the previously stored data.

5. The system of claim 4, wherein the analyzation engine is configured to apply the machine learning algorithm to calculate a likelihood that the product will be used prior to expiration of the product.

6. The system of claim 5, wherein based on the machine learning algorithm, the analyzation engine transmits a signal to the notification engine triggering the status indicator when the product associated with the RFID tag has a threshold likelihood of being used prior to expiration of the product.

7. The system of claim 6, wherein the status indicator signals a user with respect to a potential action to be performed.

8. The system of claim 7, wherein the action comprises at least one of: (i) moving the product associated with the RFID tag to different location, (ii) returning the product associated with the RFID tag to an associated manufacturer, and (iii) prioritizing utilization of the product associated with the RFID tag prior to expiration.

9. A method for managing expiration dates, the method comprising:
receiving data relating to a product associated with a radio frequency identification (RFID) tag, wherein the data corresponds to information regarding the product;
storing the received data with additional information regarding the product in a data repository;
analyzing the stored data and previously stored data; and
generating a status indicator based on the analyzation of the stored data and the previously stored data.

10. The method of claim 9, wherein the additional information includes an expiration date of the product associated with the RFID tag.

11. The method of claim 10, wherein the previously stored data includes usage data with respect to a plurality of products.

12. The method of claim 11, wherein the analyzation further comprises applying a machine learning algorithm to the stored data and previously stored data.

13. The method of claim 12, further comprising applying the machine learning algorithm to calculate a likelihood that the product will be used prior to expiration of the product.

14. The method of claim 13, further comprising, based on applying the machine learning algorithm, transmitting a signal triggering the status indicator when the product associated with the RFID tag has a threshold likelihood of being used prior to expiration.

15. The method of claim 14, wherein the status indicator signals a user with respect to a potential action to be performed.

16. The method of claim 15, wherein the action comprises at least one of: (i) moving the product associated with the RFID tag to different location, (ii) returning the product associated with the RFID tag to an associated manufacturer, and (iii) prioritizing utilization of the product associated with the RFID tag prior to expiration.

17. A non-transitory computer-readable recording medium having stored therein a program, which when executed by circuitry of a system, causes the system to:
receive data relating to a product associated with a radio frequency identification (RFID) tag, wherein the data corresponds to information regarding the product;
store the received data with additional information regarding the product in a data repository;
analyze the stored data and previously stored data; and
generate a status indicator based on the analyzation of the stored data and the previously stored data.

18. The non-transitory computer-readable recording medium of claim 17, wherein the additional information includes an expiration date of the product associated with the RFID tag.

19. The non-transitory computer-readable recording medium of claim 18, wherein the previously stored data includes usage data with respect to a plurality of products.

20. The non-transitory computer-readable recording medium of claim 19, wherein the analyzation further comprises apply a machine learning algorithm to the stored data and previously stored data.
